(19) United States
(12) Patent Application Publication
(10) Pub. No.: US 2022/0152266 A1
(43) Pub. Date: May 19, 2022

(54) DEVICE FOR PURIFICATION, STERILIZATION AND DEODORIZATION OF AIR

(71) Applicant: Liaoning Renhe Yili Environmental Protection Technology Co., Ltd., Shenyang (CN)

(72) Inventors: Zhan ZHANG, Shenyang (CN); Xiaolin ZHANG, Shenyang (CN); Li NA, Shenyang (CN); Xiaoshu SUN, Shenyang (CN)

(21) Appl. No.: 17/180,893

(22) Filed: Feb. 22, 2021

(30) Foreign Application Priority Data
Nov. 13, 2020 (CN) 202022623040.5

(51) Int. Cl. A61L 9/20; B01D 46/00; B01D 46/10; B01D 39/08; B01D 46/42; B01D 53/00; B01D 53/88; B01D 53/86; B01D 53/75; B01J 35/00; B01J 37/02; B01J 21/06; A61L 9/014; A61L 9/16

(52) U.S. Cl. CPC A61L 9/205; A61L 2209/14; B01D 46/0038; B01D 46/0036; B01D 46/0043; B01D 46/10; B01D 39/08; B01D 46/4236; B01D 53/007; B01D 53/885; B01D 53/8668; B01D 53/75; B01J 35/004; B01J 37/0225; B01J 21/063; A61L 9/014; A61L 9/16; B01D 2257/708; B01D 2257/90; B01D 2257/91; B01D 2255/20707; B01D 2255/802; B01D 2259/804; B01D 2259/818; B01D 2273/30; B01D 2279/65; A61L 2209/212; B01D 46/0028

(57) ABSTRACT

The disclosure provides a device for purification, sterilization and deodorization of air. The device includes a housing, a sterilization and disinfection device, a fan, a deodorization device, and a photocatalyst reactor. The housing is in a barrel shape and includes a side wall including a plurality of air inlets. The fan is disposed on one end of the housing. The deodorization device, the photocatalyst reactor and the sterilization and disinfection device are disposed in the housing.

### CROSS-REFERENCE TO RELATED APPLICATIONS

Pursuant to 35 U.S.C. § 119 and the Paris Convention Treaty, this application claims foreign priority to Chinese Patent Application No. 202022623040.5 filed on Nov. 13, 2020, the contents of which, including any intervening amendments thereto, are incorporated herein by reference. Inquiries from the public to applicants or assignees concerning this document or the related applications should be directed to: Matthias Scholl PC., Attn.: Dr. Matthias Scholl Esq., 245 First Street, 18th Floor, Cambridge, Mass. 02142.

### BACKGROUND

The disclosure relates to a device for purification, sterilization and deodorization of air.

Conventionally, to disinfect and sterilize the air in confined space such as central air conditioner, train carriages, and aircrafts, an air purification system is adopted. The air purification system includes a return air device, a drying device, a filtering device, an ultraviolet sterilizer, and a silver ion filter which are consecutively connected to one another. The air purification system is complex, costly, and not easy to maintain.

### SUMMARY

The disclosure provides a device for purification, sterilization and deodorization of air, the device comprising a housing, a sterilization and disinfection device, a fan, a deodorization device, and a photocatalyst reactor; the housing is in a barrel shape and comprises a side wall comprising a plurality of air inlets; the fan is disposed on one end of the housing; the deodorization device, the photocatalyst reactor and the sterilization and disinfection device are disposed in the housing.

In a class of this embodiment, the device further comprises a piece of filter cloth or filter material is detachably wrapped around the housing.

In a class of this embodiment, the photocatalyst reactor is an annular structure comprising a mesh and nano-sized titanium dioxide coating on the mesh.

In a class of this embodiment, the deodorization device is disposed between the housing and the annular photocatalyst reactor; and the deodorization device comprises activated carbon.

In a class of this embodiment, the photocatalyst reactor comprises a cavity and the sterilization and disinfection device is disposed in the cavity.

In a class of this embodiment, the sterilization and disinfection device is an ultraviolet generator, a plasma generator, an ozone generator, or a combination thereof.

In a class of this embodiment, the device further comprises an exhaust pipe disposed on the one end of the housing; wherein the fan and a silencer are disposed in the exhaust pipe.

In a class of this embodiment, the silencer comprises a sound-absorbing plate comprising sound-absorbing material; and the silencer is disposed at an air outlet of the fan in the exhaust pipe.

In a class of this embodiment, the sound-absorbing plate is in the shape of a cone, and an outer surface of the cone is oriented towards the fan.

The following advantages are associated with the device of the disclosure. When the air enters the device of the disclosure via the air inlets on the housing, the impurities and particles in the air are filtered by the filter cloth. The odor is removed by the activated carbon in the deodorization device. Thereafter, the air is further sterilized and disinfected in the photocatalyst reactor and the sterilization and disinfection device, such as photocatalyst reactor, plasma generator, ozone generator, or a combination thereof. The ozone generator has the function of sterilization, and can kill bacteria and other microorganisms in the air. The photocatalyst reactor can improve the sterilization and disinfection capacity of the sterilization and disinfection device in the presence of ultraviolet light, thus further degrading toxic and harmful gases in the air, killing a variety of bacteria, decomposing and innocuously treating the toxins released by bacteria or fungi. Through the device of the disclosure, formaldehyde, odors and bacteria are removed, and the purified air is discharged out from the exhaust pipe.

The device is easy to assemble, disassemble, and maintain, and can effectively remove PM2.5, toxic matters and bacteria in the air.

The area of the air inlets is 3-8 times of that of the air outlet, which improves the air treatment capacity and efficiency of device.

### BRIEF DESCRIPTION OF THE DRAWINGS

FIG. 1 is a schematic diagram of a device for purification, sterilization and deodorization of air in accordance with one embodiment of the disclosure; and

FIG. 2 is a sectional view taken from line A-A in FIG. 1.

In the drawings, the following reference numbers are used: 1. Filter cloth; 2. Deodorization device; 3. Exhaust pipe; 4. Silencer; 5. Flange; 6. Fan; 7. Photocatalyst reactor; 8. Ultraviolet generator; 9. Housing; 10. Plasma generator.

### DETAILED DESCRIPTION

To further illustrate, embodiments detailing a device for purification, sterilization and deodorization of air are described below. It should be noted that the following embodiments are intended to describe and not to limit the disclosure.

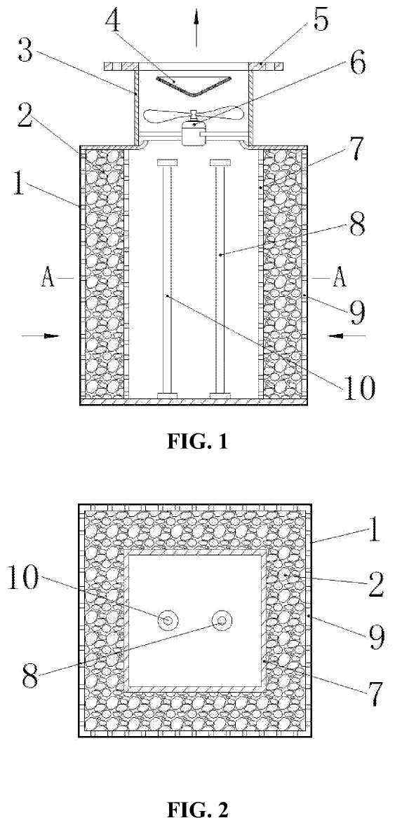

As shown in FIGS. 1-2, the disclosure provides a device for purification, sterilization and deodorization of air. The device comprises a barrel-shaped housing 9. The side wall of the barrel-shaped housing 9 comprises a plurality of air inlets. An exhaust pipe 3 is disposed on the upper part of the barrel-shaped housing 9. A fan 6 is disposed in the exhaust pipe 3. A silencer 4 is also disposed in the exhaust pipe. A deodorization device 2, a photocatalyst reactor 8, and a sterilization and disinfection device are disposed in the barrel-shaped housing 9.

A piece of filter cloth 1 or filter material is detachably wrapped around the barrel-shaped housing 9. The filter cloth is configured to block the dust with larger particles in the air.

The photocatalyst reactor 7 is an annular structure comprising a metal mesh or a non-metal mesh and nano-sized titanium dioxide coating on the mesh. The deodorization device 2 is disposed between the barrel-shaped housing 9 and the annular photocatalyst reactor 7. In certain embodiments, the deodorization device 2 comprises activated carbon.

The photocatalyst reactor 7 comprises a cavity and the sterilization and disinfection device is disposed in the cavity. The sterilization and disinfection device is an ultraviolet generator, a plasma generator, an ozone generator, or a combination thereof.

The silencer 4 comprises a sound-absorbing plate comprising sound-absorbing material. The silencer 4 is disposed at the air outlet of the fan in the exhaust pipe. The sound-absorbing plate is in the shape of a cone, and the outer surface of the cone is oriented towards the fan 6.

The flange 5 is fixed on the exhaust pipe. When in use, the device for purification, sterilization and deodorization of air is fixed and connected to an object via the flange 5. The fan is started, and a negative pressure is formed in the barrel-shaped housing 9 and the exhaust pipe 3. When the air enters the device of the disclosure via the air inlets on the barrel-shaped housing, the impurities and particles in the air are filtered by the filter cloth. The odor is removed by the activated carbon in the deodorization device. Thereafter, the air is further sterilized and disinfected in the photocatalyst reactor 7 and the sterilization and disinfection device, such as photocatalyst reactor 8, plasma generator 10, ozone generator, or a combination thereof. The ozone generator has the function of sterilization, and can kill bacteria and other microorganisms in the air. The photocatalyst reactor 7 can improve the sterilization and disinfection capacity of the sterilization and disinfection device in the presence of ultraviolet light, thus further degrading toxic and harmful gases in the air, killing a variety of bacteria, decomposing and innocuously treating the toxins released by bacteria or fungi. Through the device of the disclosure, formaldehyde, odors and bacteria are removed, and the purified air is discharged out from the exhaust pipe.

In addition, the sound-absorbing plate of the silencer 4 can prevent ultraviolet rays from being emitted from the exhaust pipe to do harm to the human body.

It will be obvious to those skilled in the art that changes and modifications may be made, and therefore, the aim in the appended claims is to cover all such changes and modifications.

What is claimed is:

1. A device, comprising a housing, a sterilization and disinfection device, a fan, a deodorization device, and a photocatalyst reactor; wherein the housing is in a barrel shape and comprises a side wall comprising a plurality of air inlets; the fan is disposed on one end of the housing; the deodorization device, the photocatalyst reactor and the sterilization and disinfection device are disposed in the housing.

2. The device of claim 1, further comprising a piece of filter cloth or filter material, wherein the piece of filter cloth or filter material is detachably wrapped around the housing.

3. The device of claim 2, wherein the photocatalyst reactor is an annular structure comprising a mesh and nano-sized titanium dioxide coating on the mesh.

4. The device of claim 3, wherein the deodorization device is disposed between the housing and the photocatalyst reactor; and the deodorization device comprises activated carbon.

5. The device of claim 4, wherein the photocatalyst reactor comprises a cavity and the sterilization and disinfection device is disposed in the cavity.

6. The device of claim 5, wherein the sterilization and disinfection device is an ultraviolet generator, a plasma generator, an ozone generator, or a combination thereof.

7. The device of claim 1, further comprising an exhaust pipe disposed on the one end of the housing; wherein the fan and a silencer are disposed in the exhaust pipe.

8. The device of claim 7, wherein the silencer comprises a sound-absorbing plate comprising sound-absorbing material; and the silencer is disposed at an air outlet of the fan in the exhaust pipe.

9. The device of claim 8, wherein the sound-absorbing plate is in the shape of a cone, and an outer surface of the cone is oriented towards the fan.
